(19) United States
(12) Patent Application Publication
(10) Pub. No.: US 2022/0314035 A1
(43) Pub. Date: Oct. 6, 2022

(54) ORAL MOUSSE COMPOSITION

(71) Applicant: BASF SE, Ludwigshafen am Rhein (DE)

(72) Inventors: Denis Ontumi, Tarrytown, NY (US); Anna Anita Gripp, Florham Park, NJ (US); Gina Marie Guiliano, Tarrytown, NY (US); Joel Basilan, Tarrytown, NY (US)

(21) Appl. No.: 17/269,112

(22) PCT Filed: Jul. 18, 2019
(86) PCT No.: PCT/US2019/042392

§ 371 Date: Feb. 17, 2021

Related U.S. Application Data
(60) Provisional application No. 62/723,089, filed on Aug. 27, 2018.

(51) Int. Cl. A61Q 11/00; A61K 8/04; A61K 8/60; A61K 8/31

(52) U.S. Cl. CPC A61Q 11/00; A61K 8/046; A61K 8/604; A61K 8/31

(57) ABSTRACT

Disclosed herein is a foam or a liquid oral care composition comprising an alkyl polyglycoside surfactant and a propellant, a method of using said oral care composition, method of its preparation, and a system comprising a pressurizable container enclosing the oral care composition.

### FIELD OF THE INVENTION

The present invention relates to the field of oral care compositions in a form of a foam or a mousse, their methods of preparation and use.

### BACKGROUND OF THE INVENTION

Most dentifrice compositions are formulated to generate foam upon brushing in the mouth. Surfactants in the formulation generate the foam. In general, the foam generated during brushing assists to uniformly disperse paste ingredients in the mouth, thereby improving the cleansing power of the toothpaste. Foam assists active agents in the toothpaste to get in between the teeth and around the gums, helping remove germs and food particles in tough to reach areas. Most toothpaste formulations are designed to start generating foam as soon as dentifrice comes in contact with saliva. The mechanical act of brushing assists with generating more foam. After one minute of brushing, all the foam has typically been generated. However, since most consumers only brush for about one minute, with the current dentifrice, the product has not fully delivered all the active ingredients to the entire oral cavity. There is a need for a product that can deliver the active ingredients to the consumer's entire oral cavity in a shorter amount of time.

### OBJECTS AND SUMMARY OF THE INVENTION

It is an object of certain embodiments of the present invention to provide an oral care composition that provides foam upon dispensing in an individual's oral cavity with or without the mechanical act of brushing.

It is another object of certain embodiments of the present invention to provide an oral care composition that accelerates the delivery of active agents in an individual's entire oral cavity immediately to improve the efficacy of the oral care composition.

It is yet another object of certain embodiments of the present invention to provide a mousse oral care product.

The above objects of the present invention and others may be achieved by the present invention which in certain embodiments is directed to a mousse oral care composition. In some embodiments, the oral care composition comprises an alkyl polyglycoside surfactant and a propellant and is in a form of a liquid or a foam.

In some embodiments, the present invention is directed to a method comprising brushing a surface in an oral cavity of a subject with an oral care composition according to an embodiment. The oral care composition may be in a form of a foam when dispensed into the oral cavity of the subject and may comprise an alkyl polyglycoside surfactant and a propellant.

In some embodiments, the present invention is directed to a method for forming an oral care composition in a liquid or a foam form. The method may comprise combining an alkyl polyglycoside surfactant and a propellant.

In some embodiments, the present invention is directed to a system comprising a sealed pressurizable container and an oral care composition enclosed in the sealed pressurizable container. The oral care composition in the sealed pressurizable container may comprise an alkyl polyglycoside surfactant and a propellant.

In some embodiments, the present invention is directed to a method of treating an oral condition. The method may comprise applying an oral care composition according to an embodiment (e.g., an oral care composition comprising an alkyl polyglycoside surfactant and a propellant) to a specified location in an oral cavity of a patient in need thereof.

### Definitions

As used herein, the singular forms “a,” “an,” and “the” include plural references unless the context clearly indicates otherwise. Thus, for example, reference to “an active agent” includes a single active agent as well as a mixture of two or more different active agents, and reference to a “propellant” includes a single propellant as well as a mixture of two or more different propellants, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number±10%, such that “about 10” would include from 9 to 11.

As used herein, the terms “active agent” and “active ingredient” refer to any material that is intended to produce a therapeutic, prophylactic, or other intended effect, whether or not approved by a government agency for that purpose. These terms with respect to specific agents include all active agents, all pharmaceutically acceptable salts thereof, complexes, crystalline forms, co-crystals, ether, esters, hydrates, solvates, and mixtures thereof.

The term “patient” refers to a subject, an animal or a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

“Pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “condition” or “conditions” refers to those medical conditions that can be treated or prevented by administration to a subject of an effective amount of an active agent, e.g., an oral condition such as plaque.

The terms “treatment of” and “treating” includes the lessening of the severity of or cessation of a condition or lessening the severity of or cessation of symptoms of a condition, e.g., an oral condition such as plaque.

The terms “prevention of” and “preventing” includes the avoidance of the onset of a condition, e.g., an oral condition such as plaque.

“Therapeutically effective amount” is intended to include an amount of an active agent, or an amount of the combination of active agents, e.g., to treat or prevent the condition, or to treat the symptoms of the condition, in a subject.

The phrase “pharmaceutically acceptable” refers to those compounds, materials, and/or compositions, within the scope of sound medical judgment, suitable for use in contact with the tissues of human beings and animals without excessive toxicity, irritation, allergic response, or other problems or complications commensurate with a reasonable benefit/risk ratio.

The term “separation” is intended to include precipitation of solids from a suspension, sol (also referred to as a colloidal solution suspension), or solution of the oral care composition and may be measured qualitatively by visual observation or quantitatively. Separation may also refer to separation of an organic phase (e.g., oil) from an aqueous phase (e.g., water).

The term “aeration” refers to a process by which a gas, e.g., air, is circulated through or dissolved in a liquid and may be measured qualitatively by visual observation (e.g., of the bubble size) or quantitatively.

The term “discoloration” refers to the change of color of an oral care composition and may be measured qualitatively by visual observation or quantitatively.

### DETAILED DESCRIPTION

According to various embodiments, the present disclosure is related to an oral care composition with a propellant and surfactant system that generates foam immediately upon dispensing into the oral cavity. The oral care composition in the form of a foam enhances a subject's ability to reach all areas in their oral cavity (including hard to reach areas) quickly and efficiently without the additional delay to generate a foam, as associated with a commercial dentifrice. Unlike a commercial dentifrice, the oral care composition disclosed herein does not require saliva or the act of brushing to assist with foam generation.

Oral care compositions according to the disclosure comprises at least one or more propellant(s) and at least one or more alkyl polyglycoside (APG) surfactant(s). In an embodiment, the oral care composition may further comprise a foaming agent such as sodium lauryl sulfate. In some embodiments, the oral care composition may further comprise regular oral care active ingredients that are used in commercial dentifrice. In some embodiments, the oral care composition may further comprise a therapeutic active agent in a therapeutically effective amount to treat or prevent an oral condition (such as plaque for instance).

Suitable propellants that may be used in the oral care compositions disclosed herein may comprise, without limitations, one or more of propane, isobutane, n-butane, nitrous oxide, and combinations thereof. In one embodiment, at least one propellant in the oral care composition comprises a mixture of at least two of propane, isobutane, and n-butane. In one embodiment, at least one propellant in the oral care composition comprises a mixture of from about 15 wt % to about 45 wt % propane, from about 10 wt % to about 40 wt % isobutane, and from about 30 wt % to about 60 wt % n-butane. In another embodiments, at least one propellant in the oral care composition comprises a mixture of from about 20 wt % to about 40 wt % propane, from about 15 wt % to about 35 wt % isobutane, and from about 35 wt % to about 55 wt % n-butane. In one embodiment, at least one propellant in the oral care composition comprises a mixture of from about 25 wt % to about 35 wt % propane, from about 20 wt % to about 30 wt % isobutane, and from about 40 wt % to about 50 wt % n-butane. An exemplary propellant that may be used has an industry designation A-70 and a mixture of about 31 wt % propane, about 23 wt % isobutane, and about 46 wt % n-butane.

In an alternative embodiment, at least one propellant in the oral care composition comprises a mixture of from about 5 wt % to about 35 wt % propane and from about 65 wt % to about 95 wt % isobutane. In a further alternative embodiment, at least one propellant in the oral care composition comprises a mixture of from about 10 wt % to about 30 wt % propane and from about 70 wt % to about 90 wt % isobutane. In yet another alternative embodiment, at least one propellant in the oral care composition comprises a mixture of from about 15 wt % to about 25 wt % propane and from about 75 wt % to about 85 wt % isobutane. Another exemplary propellant that may be used has an industry designation A-46 and a mixture of about 19 wt % propane and about 79 wt % isobutane.

Propellant(s) may be present in the oral care composition in an amount ranging from about 1 wt % to about 10 wt %, from about 3 wt % to about 8 wt %, or from about 5 wt % to about 7 wt % (calculated as the total weight of all propellants in the oral care composition divided by the total weight of the oral care composition). The quantity of propellant(s) in the composition may depend, among other factors, on the size of the pressurizable container enclosing the oral care composition.

Suitable APG surfactant(s) that may be used in the oral care compositions disclosed herein may comprise APG C8-C10, APG C10-C16, decyl glucoside, coco-glucoside, anionic APG carboxylate, sodium lauryl glucose carboxylate, lauryl glucoside, D-glucopyranose (oligomeric, C10-16 glycosides, carboxymethyl ethers, sodium salts), C12-C16 fatty alcohol glycoside, and combinations thereof. Exemplary APG surfactant(s) that may be used may have an industry designation of Plantaren® 2000 N UP/MB, Platapon® LGC Sorb, Plantaren® 1200 N UP/MB, and Plantaren® 818 UP/MB.

APG surfactant(s) may be present in the oral care composition in an amount ranging from about 0.1 wt % to about 9 wt %, from about 0.2 wt % to about 7 wt %, or from about 0.3 wt % to about 5 wt % (calculated as the total weight of all APG surfactant(s) in the oral care composition divided by the total weight of the oral care composition). The amount of APG surfactant(s) in the composition may depend, among other factors, on the resultant taste of the composition and/or on the size of the bubbles in the foam that is formed.

In some embodiments, the oral care composition disclosed herein may further comprise an additive selected from the group consisting of flavoring agent, sweeteners, active agent, colorant, dyes, antioxidants, binders, solvents, humectants (e.g., sorbitol, glycerin), viscosity modifiers (e.g., block copolymer of propylene oxide and ethylene oxide), foaming agent (e.g., sodium lauryl sulfate), solubilizers, desensitizing agent, bleaching agent, anti-cavity agents (e.g., sodium F), stain prevention agents, complexing agents (e.g., tetrapotassium pyrophosphate, tetrasodium pyrophosphate), preservatives (e.g., sodium benzoate), pH modifying agents, opacifiers, breath freshening agents, soothing agents (e.g., bisabolol), additional surfactants, additional propellants, coupling agents, and combinations thereof. In certain embodiments, the additive(s) that are included in the oral care compositions disclosed herein are natural so as to minimize irritation and/or allergic reactions to the consumer.

The total amount of all additives in the oral care composition may range from about 85 wt % to about 98 wt %, from about 87 wt % to about 95 wt %, or from about 89 wt % to about 92 wt % (calculated as the total weight of all additive(s) in the oral care composition divided by the total weight of the oral care composition). For instance, solvents and/or humectants may be present in the oral care composition at up to about 70 wt %, up to about 50 wt %, or up to about 20 wt %, collectively or individually; binders for increasing viscosity may be present in the oral care composition at from about 0.5 wt % to about 5 wt %, from about 1.5 wt % to about 4.5 wt %, or from about 2 wt % to about 4 wt %; sweeteners may be present in the oral care composition at from about 0.05 wt % to about 0.4 wt %, from about 0.1 wt % to about 0.3 wt %, or from about 0.15 wt % to about 0.25 wt %; surfactants (other than the APG surfactants described above)/solubilizers/rheology modifiers may be present in the oral care composition at up to about 8 wt %, up to about 6 wt %, up to about 4 wt %, or up to about 2 wt %, collectively or individually; flavoring agents may be present in the oral care composition at from about 1 wt % to about 2 wt %, from about 1 wt % to about 1.5 wt %, or from about 1.1 wt % to about 1.3 wt %; colorants/dyes may be present in the oral care composition at up to about 1 wt % of a 10% solution; antioxidant(s) may be present in the oral care composition at up to about 0.5 wt %; preservatives/complexing agent/stain prevention agent may be present in the oral care composition at up to about 2 wt %, up to about 1.5 wt %, or up to about 1.2 wt %. Anti-cavity agents may be present in the oral care composition at up to 0.32 wt % or a weight percent that corresponds to an amount of anti-cavity agent (e.g., fluoride) delivered to a consumer of about 600 to about 1000 ppm as regulated by the Food and Drug Administration. All weight percentages being calculated as the total weight of the specified additive(s) in the oral care composition divided by the total weight of the oral care composition.

The oral care compositions may include a flavoring agent or a mixture of flavoring agents including natural or synthetic flavorants, such as flavoring oils, flavoring aldehydes, esters, alcohols, similar materials, and combinations thereof. Flavorants may include vanillin, spearmint oil, cinnamon oil, oil of wintergreen (methylsalicylate), peppermint oil, clove oil, anise oil, eucalyptus oil, citrus oils, fruit oils and essences. Particularly preferred may be flavorants such as limonene, menthone, carvone, menthol, anethole, eucalyptus oil, eucalyptol, anethole, eugenol, cassia, oxanone, a-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), cineole, and combinations thereof.

The oral care compositions may include a sweetener capable of providing a palatable and pleasurable factor to the user, and/or capable of masking undesirable flavors present in the dosage form. Exemplary sweeteners that may be in the oral care composition may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

The oral care composition may contain a therapeutic active agent in a therapeutically effective amount to treat or prevent an oral condition. Suitable therapeutic active agents may include, without limitations, steroids, NSAIDs, a fluoride ion source (e.g., sodium F anti-cavity agent), polycarboxylate polymers, polyvinyl methyl ether/maleic anhydride (PVME/MA) copolymers, an arginine ester, a zinc ion source, a stannous ion source, delmopinol, tartar control agents, an antibacterial agent, triclosan and salts thereof, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, octapinol, nisin, a zinc ion source, a copper ion source, an essential oil, a furanone, anti-inflammatory agents, antiplaque agents, antioxidants, and a bacteriocins, and salts thereof, honokiol, vitamins, anti-attachment agents, proteinaceous agents, peptides.

Abrasives may be added to the oral care composition in some embodiments. Suitable oral care abrasive or polishing agent may be, without limitations, silica abrasives such as precipitated silicas, sodium metaphosphate, potassium metaphosphate, tricalcium phosphate, dihydrated dicalcium phosphate, aluminum silicate, calcined alumina, bentonite or other siliceous materials, particulate thermosetting resins, such as melamine, phenolic, and urea-formaldehydes, and cross-linked polyepoxides and polyesters.

Colorants and/or dyes may be added to the oral care composition in some embodiments. Suitable colorants and/or dyes may include, but not be limited to, colors such as e.g., white, black, yellow, blue, green, pink, red, orange, violet, indigo, and brown.

Antioxidants may be added to the oral care composition in some embodiments. Suitable antioxidants may include, but not be limited to, natural antioxidants such as tocopherol and tocopherol acetate.

The oral care compositions of the invention may contain a binder agent. Suitable binders include, without limitations, polyvinylpyrrolidone (PVP), marine colloids, carboxyvinyl polymers, carrageenans, starches, cellulosic polymers such as hydroxyethylcellulose, carboxymethylcellulose (carmellose), hydroxypropyl methyl cellulose and salts thereof (e.g., carmellose sodium), natural gums such as karaya, xanthan, gum arabic and tragacanth, chitosan, colloidal magnesium aluminum silicate, and colloidal silica.

The oral care compositions disclosed herein may have a pH ranging from about 5 to about 8, from about 5.5 to about 7.5, or from about 6 to about 7. The pH of the oral care composition may be measured using a pH meter by adding part of the oral care composition into a 30 ml jar, inserting a calibrated pH probe into the jar, and recording the displayed pH values. The pH of the oral care composition may be adjusted with a pH adjusting material such as, without limitations, citric acid, hydrochloric acid, sodium hydroxide, etc.

The specific gravity of the oral care compositions disclosed herein may range from about 0.9 to about 1.3, from about 1.0 to about 1.2, or from about 1.05 to about 1.15. The specific gravity of the oral care composition may be measured by using a calibrated specific gravity cup based on weight of the material and cup (the reference being water having a specific gravity of 1). A part of the oral care composition is transferred into a specific gravity cup, the specific gravity cup is covered with a cover, the scale is tared to zero, the specific gravity cup is placed on the scale, the displayed value is recorded and matched to the corresponding specific gravity table.

The oral care compositions disclosed herein may be in a liquid or in a foam form. In either form, the oral care compositions disclosed herein may illustrate no separation, no aeration, and/or no discoloration at 40° C. and 75% relative humidity for a duration of at least about 4 weeks, at least about 6 weeks, at least about 8 weeks, or at least about 13 weeks.

In some embodiments, the instant disclosure may be directed to a system comprising a sealed pressurizable container enclosing an oral care composition according to any of the embodiments disclosed herein. The sealed pressurizable container may enclose from about 250 ml to about 750 ml, from about 300 ml to about 700 ml, or from about 350 ml to about 650 ml of the oral care composition.

In some embodiments, the instant disclosure may be directed to a method of forming an oral care composition according to any of the embodiments disclosed herein. For instance, the method may comprise combining an APG surfactant(s) and a propellant(s) to form any of the oral care compositions disclosed herein in a form of a liquid or a foam. In some embodiments, the method may further comprise combining an additive(s) with the APG surfactant(s) and the propellant(s) to form any of the oral care compositions disclosed herein in a form of a liquid or a foam. The various components may be combined at the weight percentages described hereinabove.

In some embodiments, the instant disclosure may be directed to a method of using an oral care composition according to any of the embodiments disclosed herein. For instance, the method may comprise brushing a surface in an oral cavity of a subject with any of the oral care compositions disclosed herein. The surface in the oral cavity of a subject may be selected from the group consisting of teeth, gums, and tongue.

In some embodiments, the instant disclosure may be directed to a method of treating an oral condition with an oral care composition according to any of the embodiments disclosed herein. For instance, the method may comprise applying the oral care composition to a specified location in an oral cavity of a patient in need thereof.

### EXAMPLES

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Illustrative Example

### Example 1: Oral Care Compositions

In this example, five oral care compositions were prepared. Composition 1 is the control and compositions 2-5 are in accordance with an embodiment.

TABLE 1

| | | Comp. | Comp. | Comp. | Comp. | Comp. |
|---|---|---|---|---|---|---|
| Ingredients | Function | 1 | 2 | 3 | 4 | 5 |
| Water | | 55 | 53.23 | 53.23 | 53.23 | 53.23 |
| Sorbitol | Solvent, | 9 | 9 | 9 | 9 | 9 |
| | humectant, | | | | | |
| | sweetener | | | | | |
| Glycerin | Solvent and | 20 | 20 | 20 | 20 | 20 |
| | humectant | | | | | |
| Polyvinylpyrollidine | Binder | 3 | 3 | 3 | 3 | 3 |
| (Luviskol ® K 90 20% solution) | | | | | | |
| Saccharin | Sweetener | 0.2 | 0.2 | 0.2 | 0.2 | 0.2 |
| Poloxomer 407 - Pluracre 127 | Surfactant and | 2 | — | — | — | 2 |
| | rheology modifier | | | | | |
| Sodium Lauryl Sulfate | Foaming agent | 2 | — | — | — | — |
| (Texapon Z95) | | | | | | |
| Sodium F | Anti-cavity agent | 0.32 | 0.32 | 0.32 | 0.32 | 0.32 |
| Sodium Benzoate | Preservative | 0.5 | — | — | — | — |
| Wintergreens/peppermint/spearmint | Flavor agent | 1.2 | 1.2 | 1.2 | 1.2 | 1.2 |
| Blue/green/red dye - 10% sln | Colorant | 0.78 | 1 | 1 | 1 | 1 |
| Vitamin E - tocopherol | Antioxidant | — | 0.5 | 0.5 | 0.5 | 0.5 |
| (Cepherol 1300) | | | | | | |
| A70-Isobutane | Propellant | 6 | — | 6 | 3 | 6 |
| Nitrous Oxide | Propellant | — | 6 | — | 3 | — |
| APG- Plantaren ® 1200 UP | Surfactant | — | 0.4 | 0.4 | 0.4 | 0.4 |
| Bisabolol rac. | Soothing agent - | — | 0.08 | 0.08 | 0.08 | 0.08 |
| | anti irritant | | | | | |
| Tetrasodium pyrophosphate | Preservative, | — | 1.07 | 1.07 | 1.07 | 1.07 |
| | complexing agent, | | | | | |
| | stain prevention | | | | | |
| APG -Plantapon LGC Sorb | Surfactant | — | 4 | 4 | 4 | 4 |

| | | | | | |
|---|---|---|---|---|---|
| Total | 100 | 100 | 100 | 100 | 100 |
| pH value at 23° C. | 6.2 | 7 | 7 | 7 | 7 |
| Density | 1.13 | 1.12 | 1.12 | 1.12 | 1.11 |

### Example 2: Oral Care Composition Stability Data

A 13-week stability study of the five compositions from Example 1 was conducted. The compositions were analyzed after 4 weeks of aging, 6 weeks of aging, 8 weeks of aging, and 13 weeks of aging in a clear glass vial in an oven set at 40° C. and 75% relative humidity. The compositions were tested visually as to solution clarity, turbidity, and to whether any separation (i.e., observing whether the solution is homogenous or if there is a separation between an oil and a liquid phase), aeration, or discoloration was observed. No change in a particular property (e.g., separation, aeration, or discoloration) for a particular composition was designated with the number 0. A trace change in a particular property for a particular composition was designated with the number 1. A slight change in a particular property for a particular composition was designated with the number 2. A moderate change in a particular property for a particular composition was designated with the number 3. A significant change in a particular property for a particular composition was designated with the number 4. The pH of each sample was also tested.

The results of the 4 weeks aging analysis are summarized in Table 2. The results of the 6 weeks aging analysis are summarized in Table 3. The results of the 8 weeks aging analysis are summarized in Table 4. The results of the 13 weeks aging analysis are summarized in Table 5.

As seen in Tables 2-5, no visual separation, aeration, or discoloration was observed for any of the inventive compositions (Comp. 2-Comp. 5) over at least about 4 weeks, at least about 6 weeks, at least about 8 weeks, or at least about 13 weeks under storage conditions at 40° C. and 75% relative humidity. The results observed for the inventive compositions are consistent with the results observed for the control.

### Example 3: Oral Care Composition

In this example, an oral care composition were prepared according to an embodiment. First, phase A was prepared by adding Poloxomer 407 into water with gentle stirring and heating at about 50-70° C. Second, polyvinylpyrollidine powder was added to the Poloxamer and water and mixed until dissolved. Subsequently, the components of phase B were added sequentially to phase A in the listed order, followed by sequential addition of the components of phase C. Then, citric acid was added and mixed until dissolved. The resulting composition was placed in a mousse container (e.g., a pressurizable container) followed by addition of the propellant to form the final oral care composition. The pH of the final oral care composition was then measured.

TABLE 6
Oral Care Composition

| Phase | Ingredients | Function | Comp. 1 |
|---|---|---|---|
| A | Water | | 51.50 |
| | Poloxomer 407 -Pluracre 127 | Surfactant and | 2.00 |
| | | rheology | |
| | | modifier | |
| | Polyvinylpyrollidine | Binder | 3.00 |
| | (Luviskol ® K 30 solution) | | |
| B | Sodium Benzoate | Preservative | 0.50 |
| | Sodium F | Anti-cavity | 0.32 |
| | | agent | |
| | Sorbitol | Solvent, | 9.00 |
| | | humectant, | |
| | | sweetener | |
| | Sodium Saccharin | Sweetener | 0.20 |
| | Glycerin | Solvent and | 20.00 |
| | | humectant | |
| | Vitamin E - tocopherol | Antioxidant | 0.50 |
| | (Cepherol 1300) | | |
| | FD&C Blue dye No. 1 - 1% sln | Colorant | 0.78 |
| | Tetrapotassium pyrophosphate | Preservative, | 0.50 |
| | | complexing | |
| | | agent, stain | |
| | | prevention | |
| C | Wintergreens/peppermint/spearmint | Flavor agent | 1.2 |
| | APG -Plantapon LGC Sorb | Surfactant | 4.00 |
| D | Citric Acid | pH Adjustment | 0.50 |
| E | A70-Isobutane | Propellant | 6.00 |

| | |
|---|---|
| Total | 100 |
| pH value at 23° C. | 5.5 |

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. An oral care composition comprising:
an alkyl polyglycoside surfactant; and
a propellant,
wherein the oral care composition is in a liquid or a foam form.

2. The oral care composition of claim 1, wherein the propellant is present in the oral care composition in an amount ranging from about 1 wt % to about 10 wt %.

3. The oral care composition of claim 1, wherein the alkyl polyglycoside surfactant is present in the oral care composition in an amount ranging from about 0.1 wt % to about 9 wt %.

4. The oral care composition of claim 1, wherein the propellant comprises one or more of propane, isobutane, n-butane, nitrous oxide, and combinations thereof.

5. (canceled)

6. The oral care composition of claim 4, wherein the propellant comprises a mixture comprising from about 25 wt % to about 35 wt % propane, from about 20 wt % to about 30 wt % isobutene, and from about 40 wt % to about 50 wt % n-butane.

7. The oral care composition of claim 4, wherein the propellant comprises a mixture comprising from about 15 wt % to about 25 wt % propane and from about 75 wt % to about 85 wt % isobutane.

8. The oral care composition of claim 1, wherein the pH of the oral care composition ranges from about 5 to about 8.

9. The oral care composition of claim 1, wherein the specific gravity of the oral care composition ranges from about 0.9 to about 1.3.

10. The oral care composition of claim 1, further comprising an additive selected from the group consisting of flavoring agent, active agent, colorant, dyes, antioxidants, binders, solvents, humectants, viscosity modifiers, foaming agent, solubilizers, desensitizing agent, bleaching agent, anti-cavity agents, stain prevention agents, complexing agents, preservatives, pH modifying agents, sweeteners, opacifiers, breath freshening agents, soothing agents, additional surfactants, additional propellants, coupling agents, and combinations thereof.

11. The oral care composition of claim 1, wherein the oral care composition illustrates no separation, no aeration, and no discoloration at 40° C. and 75% relative humidity for at least 4 weeks.

12. (canceled)

13. The method of claim 38, wherein the specified location in the oral cavity of the subject is selected from the group consisting of teeth, gums, and tongue.

14. A method comprising:
combining an alkyl polyglycoside surfactant and a propellant to form an oral care composition in a form of a liquid or a foam.

15. The method of claim 14, wherein the propellant is present in the oral care composition in an amount ranging from about 1 wt % to about 10 wt %.

16. The method of claim 14, wherein the alkyl polyglycoside surfactant is present in the oral care composition in an amount ranging from about 0.1 wt % to about 9 wt %.

17. The method of claim 14, wherein the propellant comprises one or more of propane, isobutene, n-butane, nitrous oxide, and combinations thereof.

18.-24. (canceled)

25. The method of claim 14, wherein the oral care composition illustrates no separation, no aeration, and no discoloration at 40° C. and 75% relative humidity for at least 4 weeks.

26. A system comprising:
a sealed pressurizable container; and
an oral care composition enclosed in the sealed pressurizable container, the oral care composition comprising:
an alkyl polyglycoside surfactant; and
a propellant.

27. The system of claim 26, wherein the sealed pressurizable container encloses from about 20 ml to about 750 ml of the oral care composition.

28.-29. (canceled)

30. The system of claim 26, wherein the propellant comprises one or more of propane, isobutene, n-butane, nitrous oxide, and combinations thereof.

31.-37. (canceled)

38. A method of treating an oral condition, the method comprising applying an oral care composition to a specified location in an oral cavity of a patient in need thereof, wherein the oral care composition comprises an alkyl polyglycoside surfactant and a propellant.
